(19) United States
(12) Patent Application Publication
(10) Pub. No.: US 2006/0247315 A1
(43) Pub. Date: Nov. 2, 2006

(54) Method of treating intestinal disorders with citrulline

(75) Inventors: Sylwia Osowska-Vincent, Paris (FR); Christophe Moinard, Bourg La Reine (FR); Luc Cynober, Sceaux (FR); Marie-Emmanuelle Le Guern, Compiegne (FR); Bernard Hublot, Compiegne (FR); Paul Bernasconi, Paris (FR)

Correspondence Address: YOUNG & THOMPSON, 745 SOUTH 23RD STREET, 2ND FLOOR, ARLINGTON, VA, 22202, US

(73) Assignees: BIOCODEX, GENTILLY (FR); UNIVERSITE RENE DESCARTES PARIS 5, PARIS CEDEX 06 (FR)

(21) Appl. No.: 11/477,861

(22) Filed: Jun. 30, 2006

Related U.S. Application Data
(62) Division of application No. 10/885,574, filed on Jul. 8, 2004.

(30) Foreign Application Priority Data
Jul. 8, 2003 (FR) 03/08349

(51) Int. Cl. A61K 31/198

(52) U.S. Cl. 514/563

(57) ABSTRACT

The present invention relates to the use of L-citrulline (I) for the preparation of a medicament intended for the treatment of pathologies linked to an intestinal insufficiency.

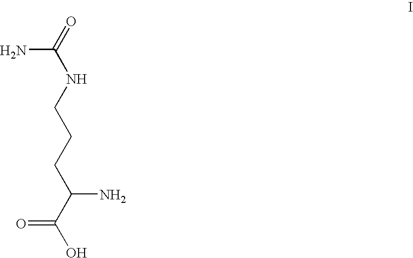

A subject of the present invention is the use of citrulline for the preparation of a medicament intended for the treatment of pathologies linked to intestinal insufficiency.

Intestinal insufficiency is defined as being a reduction of the functional intestinal mass below the quantity necessary for the absorption of nutriments (Fleming and Remington, 1981). It is characterized in particular by a lack of digestive absorption of fats, proteins, carbohydrates, or vitamins; this is then referred to as malabsorption, the repercussions of which can be malnutrition and emaciation.

One of the consequences of malabsorption is in particular to limit intakes of nitrogen and in particular of arginine. It has thus been shown in rats that in cases of massive resection of the small intestine (80%), arginine became an essential amino acid (Wakabayashi et al., 1994).

Arginine is an amino acid involved in numerous functions of the organism. It is involved in particular in the synthesis of proteins, nitrogen monoxide, polyamines, creatine and proline, moreover it possesses effects of stimulation of the secretion of hormones, such as insulin, prolactin, glucagon and growth hormone.

It is therefore crucial in the case of intestinal insufficiency to re-establish a sufficient intake of arginine, as arginine deficiencies linked to an intestinal insufficiency have such major repercussions for the organism.

The document US 2001/0056068 describes a method for treating pathologies linked to a reduction in the endogenous nitric oxide level, such as changes in intestinal motility and stenosis of the pylorus, comprising the administration of citrulline as an agent increasing the endogenous nitric oxide level. However, no mention is made of any treatment for intestinal insufficiency in this document.

Thus, one of the aims of the present invention is to provide a means for treating pathologies linked to intestinal insufficiency.

Another aim of the invention is to provide a means for re-establishing the concentration of arginine in the plasma of patients suffering from pathologies linked to an intestinal insufficiency.

The present invention results in particular from the demonstration by the Inventors that the administration by the enteral route of L-citrulline to rats that had undergone a severe resection of the small intestine made it possible to increase weight gain, increase the concentration of L-arginine in the plasma and improve the nitrogen balance, in comparison with rats that had undergone a similar resection but had not received L-citrulline. Unexpectedly, the administration of L-citrulline is more effective for increasing weight gain, increasing the concentration of L-arginine in the plasma, and improving the nitrogen balance, than is the administration of L-arginine itself.

The present invention relates to the use of L-citrulline (I) for the preparation of a medicament intended for the treatment of pathologies linked to an intestinal insufficiency.

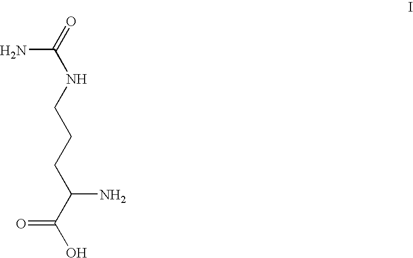

By “intestinal insufficiency” is meant a pathological state of the intestine, in particular of the small intestine, in which the absorption of nutriments is reduced relative to normal, the reduction in the absorption of nutriments being linked to a reduction in the number and/or functionality of intestinal cells capable of carrying out this absorption, this reduction in the number and/or functionality of intestinal cells being itself due either to a physical elimination of these cells (in particular by surgery or by radiation), or to a pathological dysfunction of these cells.

It should be noted in this connection that changes in intestinal motility, as well as stenosis of the pylorus do not form part of the field of the invention.

In fact, changes in intestinal motility are present in functional pathologies such as constipation, irritable bowel syndrome, or functional diarrhoea. These pathologies are determined neither by a reduction in the number of intestinal cells capable of carrying out absorption functions, nor by a dysfunction of these cells (Isselbacher et al., 1995; Dapoigny et al., 2003).

Moreover, stenosis of the pylorus is a relatively frequent disease in infants. It is a congenital malformation in which the pylorus is hypertrophied and constitutes a mechanical obstacle to the passage of food. It is treated surgically, by means of a simple and rapid pyloromyotomy, after which the child can very quickly resume normal feeding. It is therefore a pathology unrelated to an intestinal insufficiency (Jacqmarcq et al., 2004; Rambaud and Bouhnik, 2001).

One of the frequent consequences of intestinal insufficiency is that it leads to a state of malnutrition. The use of L-citrulline makes it possible in particular to improve the nutritional and weight condition of individuals suffering from intestinal insufficiency.

The present invention relates in particular to the above-mentioned use of L-citrulline for the preparation of a medicament intended to increase the concentration of L-arginine in the plasma when it is abnormally low in patients suffering from pathologies linked to an intestinal insufficiency.

The present invention relates more particularly to the above-mentioned use of L-citrulline for the preparation of a medicament intended for the treatment of the following pathologies:

short-bowel syndrome following an intestinal resection, in particular in the case of acute mesenteric ischaemia, thrombosis of the superior mesenteric vein, volvulus of the small intestine and strangulated hernias, chronic intestinal pseudo-obstruction, radiation-damaged small intestine, Crohn's disease, nongranulomatous ulcerous jejuno-ileitis, abdominal traumatism; short-bowel syndrome results in particular from resections of the small intestine leaving a maximum of 1 metre of small intestine besides the duodenum; these resections lead in the immediate post-operative period to an intestinal insufficiency characterized by constant and major malabsorption, sometimes aggravated by gastric hypersecretion, which leads to the setting up of total parenteral nutrition, rapidly combined with continuous enteral nutrition, then to oral feeding; the adaptation of the remaining intestine is possible between 2 and 6 months after the procedure, but the improvement in the absorption capacities of the small intestine most often remains insufficient (Rambaud and Bouhnik, 2001; Colombel and Dupas, 1997);

celiac disease; celiac disease is a chronic enteropathy characterized by a food intolerance to gluten, and more particularly to proteins contained in certain cereals, such as gliadin, hordein or secalin; this disease occurs in genetically predisposed subjects; the intestinal mucosa of a patient suffering from celiac disease is the seat of an inflammatory process, partly of an immune nature, which causes in particular atrophy of the villi; the resultant intestinal insufficiency is characterized by intestinal malabsorption, which manifests itself in diarrhoea with steatorrhoea, emaciation and malnutrition; the biological consequences of malabsorption are in particular anaemia associated with an iron, folate or vitamin B12 deficiency, a deficit of vitamin K-dependent coagulation factors, hypoproteinaemia, hypoalbuminaemia, hypocalcaemia, hypomagnesaemia and zinc deficit (Cellier and Grosdidier, 2001; Jadoulle, 2002);

chronic inflammatory diseases of the intestine, such as Crohn's disease and ulcerous rectocolitis, also called haemorrhagic rectocolitis; these diseases are chronic diseases evolving by periodic onsets, they are often accompanied, in particular in the case of Crohn's disease, by chronic intestinal insufficiency caused by fistulas, mechanical obstacles in the small intestine, or non-stenosing enteropathy; the resulting reduction in the functional intestinal mass does not allow the maintenance of the nutritional state; in fact this results in protein-energy malnutrition, which is accompanied by malabsorption and an increase in hydro-electrolytic losses and losses of mineral elements, such as calcium, magnesium or zinc, which frequently leads to malnutrition (Rambaud and Bouhnik, 2001; Crenn et al., 2001);

intestinal insufficiency linked to ageing; protein-energy malnutrition is frequent in the elderly; in fact approximately 40% of those aged over 70 years are affected; in a malnutrition situation, ageing is characterized by morphological and functional modifications of the small intestine; these changes can lead to malabsorption and aggravate the pre-existing malnutrition; moreover this malnutrition aggravates changes in the digestive system linked to age; besides, the degradation of the nutritional state furthers the risks of infections; finally, it is stated that the malnourished elderly subject presents a lack of response to the resumption of standard feeding compared with what is observed in an adult;

intestinal insufficiency linked to irradiation, also called radiation-damaged small intestine; in this case intestinal insufficiency is caused by the irradiation of the small intestine, in particular in the case of radiotherapies, and more particularly within the framework of the treatment of cancers developing in the abdominal-pelvic cavity.

According to another embodiment, the present invention relates to the above-mentioned use of L-citrulline for the preparation of a pharmaceutical composition comprising, as active substance, L-citrulline, or a pharmaceutically acceptable salt thereof, in combination with a pharmaceutically acceptable vehicle.

By “pharmaceutically acceptable salt” is meant in particular citrulline salts such as citrulline malate.

The pharmaceutically acceptable vehicles will be clearly apparent to a person skilled in the art.

The invention relates in particular to the above-mentioned use of L-citrulline for the preparation of a pharmaceutical composition characterized in that the unit L-citrulline dose is approximately 1 g to approximately 10 g, in particular approximately 5 g, for a dosage of approximately 0.05 g/kg/day to approximately 0.50 g/kg/day, in particular approximately 0.25 g/kg/day.

The invention relates more particularly to the above-mentioned use of L-citrulline for the preparation of a pharmaceutical composition presented in dry form or in the form of an aqueous solution.

The invention relates more particularly to the above-mentioned use of L-citrulline for the preparation of a pharmaceutical composition presented in a form which can be administered by the oral, intraperitoneal, enteral or parenteral route.

Administration by the enteral route corresponds in particular to an administration by gastric or intestinal probe, administration by the parenteral route corresponds in particular to administration by central, peripheral or subcutaneous intravenous perfusion.

The invention relates more particularly to the above-mentioned use of L-citrulline for the preparation of a pharmaceutical composition also containing one or more other compounds intended for the treatment of intestinal insufficiency, such as glutamine, ornithine, growth hormone, or somatomedin C.

Growth hormone, also called somatotrophin, is advantageously in recombinant form, somatomedin C, also called IGF-1 standing for insulin-like growth factor, is also advantageously in recombinant form.

According to another embodiment the present invention relates to a pharmaceutical composition characterized in that it comprises, as active substance, L-citrulline, or a pharmaceutically acceptable salt thereof, in combination with at least one other compound intended for the treatment of intestinal insufficiency, such as the compounds defined above, and with a pharmaceutically acceptable vehicle.

According to another embodiment the invention relates to products comprising:
L-citrulline,
and at least one other compound intended for the treatment of intestinal insufficiency, such as the compounds defined above,

as a combined preparation for simultaneous, separate or sequential use, in the treatment of intestinal insufficiency.

### DESCRIPTION OF THE FIGURES

FIG. 1

FIG. 1 represents the weight gain in grams (on the y-axis) of rats that have undergone an intestinal resection and are receiving standard enteral nutrition (AANE), standard enteral nutrition supplemented with arginine (ARG), standard enteral nutrition supplemented with citrulline (CIT), or have not undergone resection (SHAM) after 10 days of treatment. The estimated measurement error is shown on the top of each bar.

FIG. 2

FIG. 2 represents the concentration of arginine in the plasma in μmol/L (on the y-axis) of rats that have undergone an intestinal resection and are receiving standard enteral nutrition (AANE), standard enteral nutrition supplemented with arginine (ARG), standard enteral nutrition supplemented with citrulline (CIT), or have not undergone resection (SHAM) after 10 days of treatment. The estimated measurement error is shown on the top of each bar.

FIG. 3

FIG. 3 represents the cumulative nitrogen balance in grams (on the y-axis) of rats that have undergone an intestinal resection and are receiving standard enteral nutrition (AANE, black bar), standard enteral nutrition supplemented with arginine (ARG, vertically hatched bar), standard enteral nutrition supplemented with citrulline (CIT, obliquely hatched bar) or have not undergone resection (SHAM, white bar), for each of the 10 days of the treatment, from 1 to 10 (on the x-axis). The estimated measurement error is shown on the top of each bar.

FIG. 4

FIG. 4 represents the protein content of the Tibialis muscle in mg/organ (on the y-axis) of elderly control rats (AL), of elderly malnourished rats (R), of elderly malnourished rats that haste received food supplemented with non-essential amino acids (AANE) and elderly malnourished rats that have received food supplemented with L-citrulline (CIT) (on the x-axis). The asterisk indicates that the value measured for the CIT group is significantly different from those measured for the AL, AANE and R groups.

FIG. 5A and FIG. 5B

FIG. 5A represents the relative protein synthesis rate (FSR) in the Tibialis muscle in percentages per hour (on the y-axis) for elderly control rats (AL), elderly malnourished rats (R), elderly malnourished rats that have received food supplemented with non-essential amino acids (AANE) and elderly malnourished rats that have received food supplemented with L-citrulline (CIT) (on the x-axis).

FIG. 5B represents the absolute protein synthesis rate (ASR) in the Tibialis muscle in mg of proteins per hour (on the y-axis) of elderly control rats (AL), elderly malnourished rats (R), elderly malnourished rats that have received food supplemented with non-essential amino acids (AANE) and elderly malnourished rats that have received food supplemented with L-citrulline (CIT) (on the x-axis). The asterisk indicates that the value measured for the CIT group is significantly different from those measured for the AANE and R groups.

### EXAMPLE 1

Demonstration of the Effect of Food Supplemented with Citrulline within the Framework of Intestinal Insufficiency Applied to a Short-Bowel Syndrome Model

The rat was chosen as model to evaluate the effect of supplementation with citrulline within the framework of an intestinal insufficiency and in particular within the framework of the short-bowel syndrome.

24 Wistar rats weighing approximately 220-230 g were used. They were acclimatized in metabolic cages for 5 days before being operated on; during this period they had free access to water and standard laboratory food.

The animals were then operated on: 18 of them underwent an intestinal resection of approximately 80% of the total length of the small intestine, the last 6 rats were operated on but did not undergo resection.

4 groups of animals were formed:
an AANE group, comprising 6 animals that had undergone resection and received standard enteral nutrition (Sondalis®, Nestlé Clinical Nutrition),
an ARG group, comprising 6 animals that had undergone resection and received standard enteral nutrition supplemented with a dose of L-arginine (Sigma-Aldrich) of 0.994 g/kg/day;
a CIT group, comprising 6 animals that had undergone resection and received standard enteral nutrition supplemented with a dose of L-citrulline (Laboratoires Biocodex) of 1 g/kg/day (i.e. in a quantity equimolar to arginine),
a SHAM control group, comprising 6 animals that had been operated on but had not undergone a resection, and were receiving standard enteral nutrition.

The intakes of L-arginine and L-citrulline correspond to those used in humans taking account of the difference in metabolic rate between the two species (rate approximately 10 times higher in the rat).

Enteral nutrition was carried out for 10 days continuously by the insertion of a gastric probe. The nitrogen intake was 2 g/kg/day and the calorie intake 290 kcal/kg/day. The food rations were rendered isonitrogenous by intake of a mixture of non-essential amino acids (Gly, Ala, Ser, His, Pro, Asn) (Sigma-Aldrich).

Three types of measurements were carried out in particular: a daily measurement of the weight of the animals, a measurement of the concentration of arginine in the plasma at the end of the experiment by ion-exchange chromatography using a JEOL device and a daily monitoring of the cumulative nitrogen balance by adding up the daily differences between the quantity of nitrogen absorbed and the quantity of nitrogen evacuated in the urine by pyroluminescence using an ANTEK apparatus.

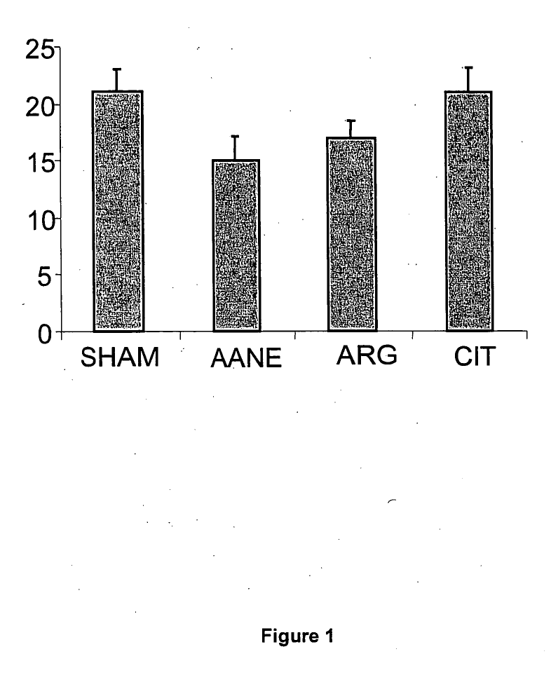

The results shown in FIG. 1 indicate that the weight gain of the AANE group after 10 days (approximately 15 g) is less than that of the SHAM control group (approximately 21 g); on the other hand the weight gain of the ARC group (approximately 17 g) is greater than that of the AANE group but less than that of the SHAM control group, and the weight gain of the CIT group (approximately 21 g) is equivalent to that of the SHAM control group. Supplementation with citrulline therefore makes it possible to compensate for the reduction in weight gain due to resection, which indicates that in cases of intestinal insufficiency, the administration of citrulline makes it possible to maintain the nutritional state of the animals.

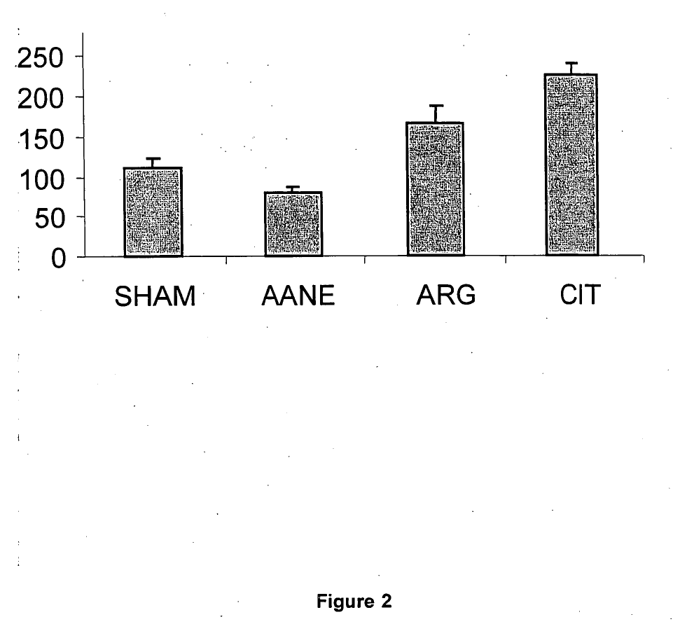

The results shown in FIG. 2 indicate that the concentration of arginine in the plasma is lowered for the AANE group compared with the SHAM control group, which confirms the data of the prior art concerning arginine deficiency resulting from a severe intestinal resection. On the other hand supplementation with arginine (ARG group) makes it possible to compensate for this effect, and even to increase the concentration of arginine in the plasma compared with the control. This effect is even more marked in the case of citrulline (CIT group).

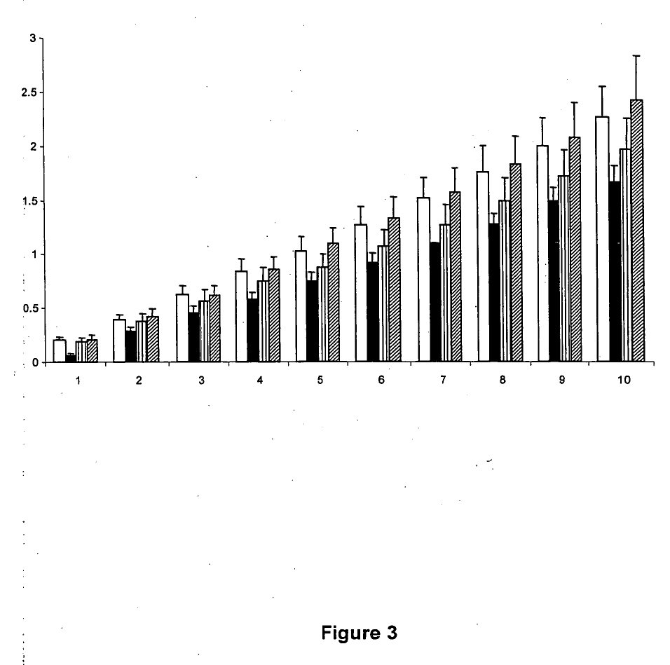

However, the results shown in FIG. 3 indicate that only supplementation with citrulline (CIT group) makes it possible to re-establish a nitrogen balance in the animals that have undergone resection, at the same level as that of the control group (SHAM), whereas this is not the case with supplementation with arginine (ARG group).

Overall these results indicate that in cases of intestinal insufficiency citrulline can (i) make it possible to maintain the nutritional state, (ii) compensate for the effects of arginine malnutrition by restoring a normal nitrogen balance and (iii) be absorbed by the intestine in quantities sufficient to allow it to exert its effects.

### EXAMPLE 2

Demonstration of the Effect of Food Supplemented with Citrulline within the Framework of Intestinal Insufficiency Linked to Ageing.

The rat was chosen as model to evaluate the effect of supplementation with L-citrulline within the framework of the treatment of an intestinal insufficiency linked to ageing.

All the chemical reagents used come from Sigma (Saint-Quentin-Fallavier, France). The L-citrulline was supplied by Laboratoires Biocodex (Compiègne, France).

Male Sprague-Dawley rats (Charles River Laboratories, L'Arbresles, France) aged 19 months were placed in individual cages, in a thermostatically controlled ambience (23°±1° C.), and subjected to a 12-hour light-darkness cycle (darkness from 08.00 hours to 20.00 hours). Their acclimatization was carried out over 2 weeks, during which their spontaneous food consumption was measured. They were fed a standard diet (A04, UAR, Villemoisson-sur-Orge, France) containing 17% proteins, 3% lipids, 59% carbohydrates and 21% water, fibres, vitamins and minerals. The average food intake during this period is 34.4 g/day.

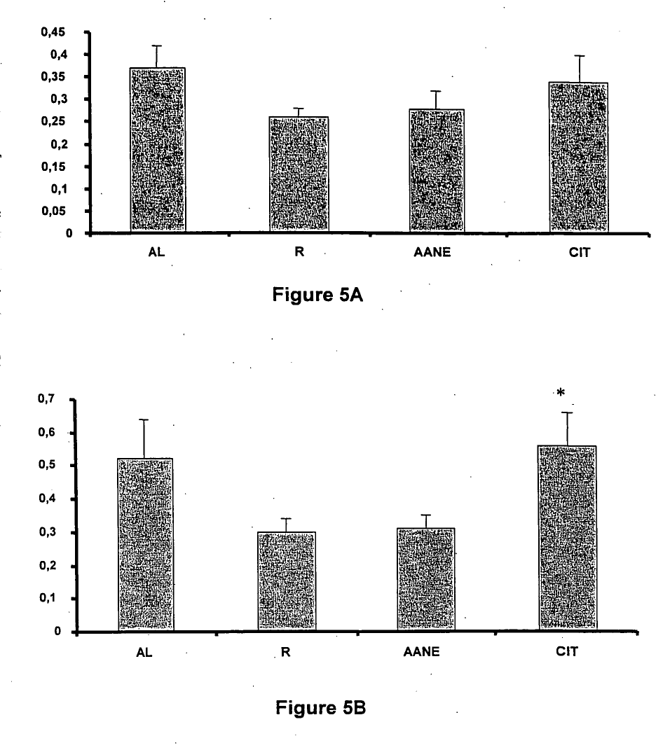

After the acclimatization period, the rats were divided into 4 groups (cf. FIG. 5): a control group comprising rats (n=10) fed ad libitum (AL) for 12 weeks and 3 other groups subjected to a food restriction during the same period, i.e. fed with only 50% of the spontaneous ingesta (i.e. 17.2 g), with the standard diet (UAR A04).

At the end of the restriction period, the animals (n=10) in one group were sacrificed (group R) and the rats of the two remaining groups were fed for a week with 90% of the spontaneous ingesta (i.e. 30.9 g), either on a diet enriched with non-essential amino acids (AANE group: alanine, asparagine, glycine, serine, histidine, and proline supplied in equimolar fashion) or on a diet enriched with L-citrulline (5 g/kg/d) (citrulline group). The intakes of the two groups were isonitrogenous and isocaloric. The limitation to 90% of the spontaneous intakes makes it possible to be certain that the rats consume all of the food offered to them (Walrand et al., 2000).

Body weight was measured every three days throughout the experimentation period.

At the end of the experimental period, the rats, in post-absorptive situation, were anaesthetized with isoflurane (3%) and received a subcutaneous injection of ¹³C-valine at variable times before being killed by decapitation.

The 2 Tibialis muscles (TIB) were removed in order to determine on the one hand the protein and amino acid content (right TIB) and on the other hand, the fractional (FSR) and absolute (ASR) protein synthesis rates (left TIB). All the tissues taken were weighed beforehand, before being frozen in liquid nitrogen and stored at −80° C. until the final analyses.

The blood was collected on heparin and centrifuged (3500 rpm, at +4° C. for 15 minutes). Then, the plasmas was deproteinized using 100 μL of a 30% sulphosalicylic acid solution per 1 mL of plasma. The samples were centrifuged (5000 rpm, +4° C., 5 minutes). An aliquot fraction of the supernatant was assayed by cation-exchange chromatography on an amino acids analyzer (Jeol, Tokyo, Japan). The results were expressed in μmol/L.

Determination of the protein content was carried out on the TIB. The frozen tissues were ground and homogenized in a 10% trichloroacetic acid (TCA) solution (1 mL per 100 mg of tissues) using an Ultra-Turrax T25® grinder (Ika Labotechnik, Staufer, Germany), while keeping the tube in the ground ice. The homogenate was then centrifuged for 10 minutes at 3500 rpm. Then, the ground pellet was delipidated with an ethyl alcohol/ether (v/v) mixture, and dissolved in 1N soda (4 mL per 100 mg of tissues) for 12 hours at 40° C. The proteins content of the TIB is measured by the Gornall method. The results are expressed in mg/muscle.

Determination of the amino acids content was canned out on the Tibialis. In order to do this, the intratissue amino acids were extracted before their assay. For this purpose, the muscle considered was ground in the presence of a 10% TCA (trichloroacetic acid) solution (1 ml per 100 mg of tissue) in a glass tube immersed in ice. The grinding was carried out with an Ultra-Turrax® apparatus, until a homogeneous suspension was obtained. After centrifugation (10 minutes at 3500 rpm at +4° C.), the supernatant, which contains the free amino acids, was divided into aliquot fractions and frozen at −80° C. until the assay. The latter was carried out by cation-exchange chromatography, using an Aminotac model Jeol analyzer, (Tokyo, Japan). The results are expressed in nmol/g of tissue.

The synthesis rate was determined on the TIB by the method of injecting a dose of a tracer amino acid charge. The principle of the technique rests on the detection by mass spectrometry of the incorporation of this tracer amino acid in the proteins of interest (Guillet et al., 2004). In practice, the ¹³C-valine (150 μmol/100 g of body weight, Cambridge Isotope Laboratories, MA, USA) was injected by the subcutaneous route (3.6 mL per 600 g of body weight). Then, in order to take into account the rate of incorporation of the tracer into the proteins, the animals of the same group were killed at different times: 10, 15, 20, 25, 30, 35, 40, 45, 50 and 55 minutes post injection. However, as the numbers in the AL (n=8) and R groups (n=9) made it impossible to include all the times, it was necessary to exclude 2 experimental points from the AL group (t=45 and 55 minutes) and 1 point from the R group (t=55 minutes). This made it possible to determine the incorporation of the tracer in the proteins of interest and to establish a straight line of incorporation (Breuille et al., 1998). After grinding of 100 mg of muscle, the proteins were extracted by successive precipitations in trichloroacetic acid (TCA). They were then hydrolyzed with HCl (6M, 110° C., 24 hours) and the amino acids thus obtained were purified by cation-exchange chromatography (Dowex 50W 8X, Bio-Rad, Hercules, Calif.). The amino acids were eluted with soda (4M) and speedvac-dried (Savant Instrument Inc, USA). They were then converted to N-acetylpropyl derivatives. Moreover, the ¹³C-valine enrichment in the muscle free amino acids compartment was also measured by mass spectrometry coupled with gas-phase chromatography (GC-MS type, Hewlett-Packard 5971A, USA), after extraction of the amino acids with perchloric acid and derivatization to t-butyldimethylsilyl esters.

The fractional myofibrillar protein synthesis rate (FSR) expressed in % per hour was calculated for each point taking into account both the ¹³C-valine enrichment of the muscle proteins, but also that of the synthesis precursor compartment (pool of free amino acids in the muscles). Thus, the following formula is used:
FSR=[(Sb−Sb0)/(Sa×t)]×100
where:

Sb: ¹³C-valine enrichment of the muscle proteins at time t;

Sb0: natural ¹³C-valine enrichment of the muscle proteins

Sa: ¹³C-valine enrichment in the intra-muscle free amino acids compartment;

t: post-injection time of sampling of the muscle in hours.

This parameter is also expressed as an absolute quantity of muscle proteins synthesized per hour (ASR, in mg/h) talking into account the total quantity of proteins within the TIBs of each rat, according to the equation:
ASR=[FSR×quantity of protein in mg]/100

The results are presented in the form of an average±standard deviation from the mean (SDM). The results are analyzed by an ANOVA followed by an a posteriori Duncan test. PCSM software is used (Deltosoft, Grenoble, France). The values of p<0.05 are regarded as significant.

The results obtained indicate that only supplementation with L-citrulline significantly increases concentrations of CIT, ORN and ARG (CIT vs AL, R, AANE, p<0.05) in the plasma and muscles (Table 1).

TABLE 1
Concentrations of amino acids in the plasma and the TIB

| | | | |
|---|---|---|---|
| AL | R | AANE | CIT |

Plasma

| | | | | |
|---|---|---|---|---|
| Citrulline | 116 ± 7 | 104 ± 7 | 155 ± 20 | 2394 ± 279* |
| Ornithine | 46 ± 3 | 41 ± 3 | 43 ± 6 | 223 ± 27* |
| Arginine | 114 ± 9 | 118 ± 4 | 99 ± 7 | 561 ± 55* |

TIB

| | | | | |
|---|---|---|---|---|
| Citrulline | 23 ± 4 | 26 ± 2 | 50 ± 11 | 616 ± 104* |
| Ornithine | 4 ± 0 | 6 ± 0 | 3 ± 0 | 20 ± 4* |
| Arginine | 29 ± 3 | 66 ± 4 | 22 ± 3 | 203 ± 35* |

AVERAGE ± SDM. The results are expressed in μmol/L and nmol/g respectively
ANOVA+ Duncan test: *p < 0.05 vs AL, R and AANE

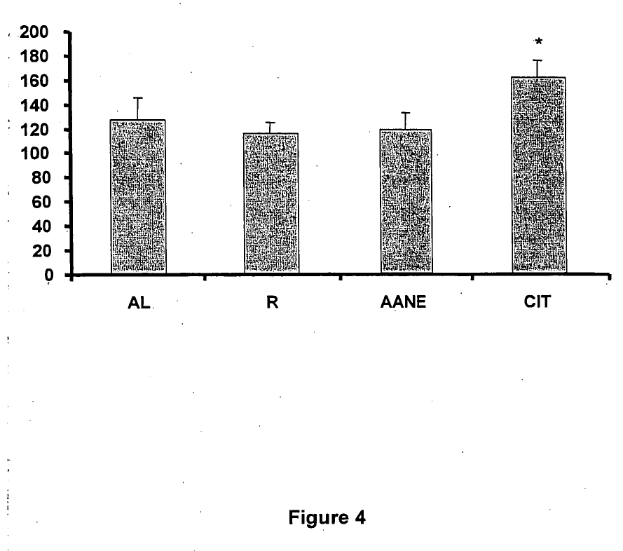

Moreover, the food restriction does not lead to a significant reduction in the protein content of the muscle. However, following the renutrition phase, a significant increase in the protein concentrations in the TIB is observed only for the CIT group (CIT vs AL, R, AANE, p<0.05) (FIG. 4).

Finally, although greater for L-citrulline compared with the other groups, the variation in the FSR observed does not seem significant (FIG. 5A). On the other hand, there is a significant increase in the ASR only for the group receiving L-citrulline-enriched nutrition (CIT vs R and AANE, p<0.05) (FIG. 5B).

As a result, supplementing the diet of malnourished aged rats with L-citrulline makes it possible to obtain a significant increase in the muscular mass and protein accretion, in particular at the level of the Tibialis. This indicates that L-citrulline is suitable for the treatment of intestinal insufficiency linked to ageing.

### REFERENCES

Breuille D et al. Clin. Sci. (1998) 94: 413-423
Cellier C and Grosdidier E. Maladie Coeliaque de l'Adulte. Rev. Prat. (2001) 51: 959-963.
Colombel J F and Dupas J L. Pathologie du Grêle. In: Progrès en Hépato-gastroentérologie. Paris, Doin, Ed. (1997) 219-220.
Crenn P et al. Insuffisance Intestinale. Rev. Prat. (2001) 51: 977-982.
Dapoigny et al. Troubles fonctionnels intestinaux et consommation de soins. Gastroentérologie Clinique et Biologique, (2003) 27: 265-271.
Fleming C R and Remington M. Intestinal Failure. In: Nutrition and the Surgical Patient: Clinical Surgery International. Hill G I, Ed. Edinburgh: Churchill Livingstone, (1981) 219-235.
Guillet C et al. Exp. Gerontol. (2004) 39: 745-751
Isselbacher K J et al. Trouble de la motricité intestinale. In: Harrison Médecine Interne. Arnette Blackwell S. A. (13^{th} edition) (1995) 1418-1422.
Jacqmarcq et al. Prise en charge périopératoire de la sténose du pylore en France en 1999. Annales franaises d'Anesthésie et de Réanimation. (2004) 23: 31-38.
Jadoulle J. Interactions Somato-Psychiques dans la Maladie Coeliaque. Gastroentérol. Clin. Biol. (2002) 26: 1134-1139.
Wakabayashi Y et al. Arginine Becomes an Essential Amino Acid after Massive Resection of Rat Small Intestine. J. Biol. Chem. (1994) 269: 32667-32671.
Rambaud J C auld Bouhnik Y. Intestin Grêle. In: Le Livre de l'Interne en Gastroentérologie. Paris: 2^{nd} edition, Flamarion, Ed. (2001) 286-298, 533-536.
Rambaud J C and Bouhnik Y. sténose hypertrophique du pylore. In: Le Livre de l'Interne en Gastroentérologie. Paris: 2^{nd} edition, Flamarion, Ed. (2001) 202.
Walrand S et al. Am. J. Clin. Nutr. (2001) 74: 670-678

1. A method for the treatment of pathologies linked to an intestinal insufficiency, comprising the administration of a pharmaceutically acceptable amount of L-citrulline (I):

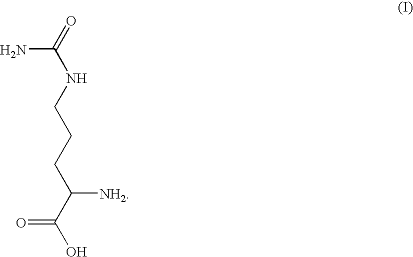

2. The method of treatment according to claim 1, wherein the concentration of L-arginine in the plasma is increased when it is abnormally low in patients suffering from pathologies linked to an intestinal insufficiency.

3. The method of treatment according to claim 1, wherein the pathologies are selected from the group consisting of:
short-bowel syndrome following an intestinal resection,
celiac disease,
chronic inflammatory diseases of the intestine,
intestinal insufficiency linked to ageing, or
intestinal insufficiency linked to irradiation.

4. The method according to claim 11, comprising the administration as active substance, L-citrulline, or a pharmaceutically acceptable salt thereof, in combination with a pharmaceutically acceptable vehicle.

5. The method according to claim 1, wherein L-citrulline is administered at a dosage of approximately 0.05 g/kg/day to approximately 0.50 g/kg/day.

6. The method according to claim 1, wherein L-citrulline is presented in dry form or in the form of an aqueous solution.

7. The method according to claim 1, wherein L-citrulline is presented in a form which can be administered by the oral, intraperitoneal, enteral or parenteral route.

8. The method according to claim 1, also comprising the administration of one or more other compounds chosen from the group consisting of glutamine, ornithine, growth hormone, or somatomedin C.

9. A method for treating an intestinal insufficiency in a patient, comprising administering to said patient an effective amount of L-citrulline (I):

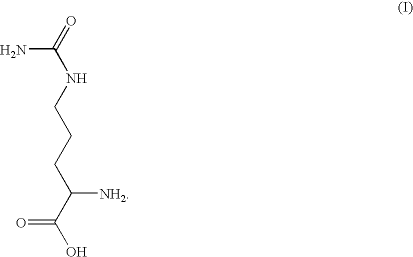

10. The method according to claim 9, wherein the subject suffers an intestinal insufficiency selected from the group consisting of:
short-bowel syndrome,
celiac disease,
chronic inflammatory diseases of the intestine,
intestinal insufficiency linked to ageing, or
intestinal insufficiency linked to irradiation.

11. The method according to claim 9, comprising the administration as active substance, L-citrulline, or a pharmaceutically acceptable salt thereof, in combination with a pharmaceutically acceptable vehicle.

12. The method according to claim 9, wherein L-citrulline is administered at a dosage of approximately 0.05 g/kg/day to approximately 0.50 g/kg/day.

13. The method according to claim 9, wherein L-citrulline is in an aqueous-solution.

14. The method according to claim 9, wherein L-citrulline is administered by the oral, intraperitoneal, enteral or parenteral route.

15. The method according to claim 9, further comprising administering one or more other compounds selected from the group consisting of glutamine, ornithine, growth hormone, or somatomedin C.

16. A method for treating a patient having short-bowel syndrome, comprising administering to said patient an effective amount of L-citrulline (I):

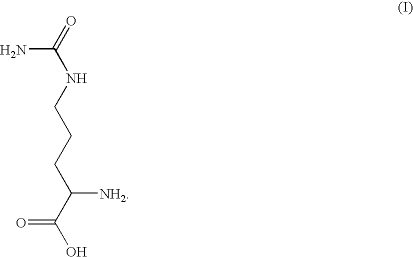

17. The method according to claim 16, wherein L-citrulline is administered at a dosage of approximately 0.05 g/kg/day to approximately 0.50 g/kg/day.

18. The method according to claim 16, wherein L-citrulline is administered in an aqueous solution.

19. The method according to claim 16, wherein L-citrulline is administered orally.

20. The method according to claim 9, wherein said patient suffers from an intestinal insufficiency due to aging.
